(19) United States
(12) Patent Application Publication
(10) Pub. No.: US 2025/0302788 A1
(43) Pub. Date: Oct. 2, 2025

(54) COMPOSITION COMPRISING CREATINE FOR USE IN THE TREATMENT OF POST VIRAL FATIGUE SYNDROME

(71) Applicant: Alzchem Trostberg GmbH, Trostberg (DE)

(72) Inventors: Jürgen BEZLER, Burghausen (DE); Verena LÖBER, Truchtlaching (DE); Robert ALBER, Prien (DE); Sergej M. OSTOJIC, Pancevo (RS)

(73) Assignee: Alzchem Trostberg Gmbh, Trostberg (DE)

(21) Appl. No.: 18/864,432

(22) PCT Filed: May 11, 2023
(86) PCT No.: PCT/EP2023/062671

§ 371 Date: Nov. 8, 2024

(30) Foreign Application Priority Data
May 12, 2022 (EP) 22172901.5
Jul. 28, 2022 (EP) 22187487.8
Feb. 3, 2023 (EP) 23154842.1
Feb. 3, 2023 (EP) 23154844.7

(51) Int. Cl. A61K 31/198; A61K 31/7004; A61P 43/00

(52) U.S. Cl. CPC A61K 31/198; A61K 31/7004; A61P 43/00

(57) ABSTRACT

The present invention relates to a pharmaceutical composition comprising creatine or physiologically acceptable derivatives and/or salts and or adducts thereof for use in the treatment of PVFS, in particular associated with mental fatigue, reduced motivation, reduced activity, or concentration difficulties. A further embodiment of the invention relates to the use of a composition comprising creatine or physiologically acceptable derivatives and/or salts and or adducts thereof as dietary supplement for preparation of a diet supporting the recovery from PVFS.

### FIELD OF THE INVENTION

The present invention is related to pharmaceutical compositions comprising creatine for use in the treatment of post viral fatigue syndrome (PVFS) caused by virus infection of the lungs or the lower respiratory system in a subject in need thereof. A further object of the invention is the use of creatine or creatine derivatives as dietary supplement for the preparation of a diet for patients suffering under PVFS.

### DESCRIPTION OF THE PRIOR ART

Although COVID-19 is seen as a disease that primarily affects the lungs, it can also damage other organs, including the heart, the vascular system, the kidneys and the brain for example. Organ damage increases the risk of sequelae, such as cognitive impairment, heart complications (myocarditis), chronic kidney impairment, stroke, thrombosis, and Guillain-Barre syndrome.

However, most people who are infected with SARS-COV-2 (Severe Acute Respiratory Syndrome coronavirus type 2) recover within a few weeks. But some people, even those who had mild versions of the disease, continue to experience a brought variety of symptoms after their initial recovery.

Typical symptoms that may persist after a SARS-COV-2 infection are for example fatigue (e.g. post-viral fatigue syndrome (PVFS) or chronic fatigue syndrome (CFS)), breathing difficulties or breathlessness (dyspnea), lung or chest pain, joint pain, muscle pain or headache, concentration and memory difficulties, sleep problems (insomnia), loss of smell (anosmia) or taste (ageusia) etc., wherein fatigue, breathing difficulties (dyspnea) and chest pain belongs to the most reported conditions after acute SARS-COV-2 infection.

PVFS is a complex long-term disorder, characterized by an inability to participate in routine activities in daily life that were possible before the virus infection, which lasts for at least 3 months. Besides other negative consequences, coronavirus infections are often linked with PVFS.

In serious courses of disease, about 70% of COVID-patients suffer from fatigue in the acute phase of the infection, wherein the symptom often persists after recovery of the patients from infection (Manal S., Barnett J., Brill S. et al. (Thorax, 2021; 76, 396-398), Halpin S. J., Mclvor C., et al. (J. Med. Virol. 2021; 93, 1013-1022, Carfi A., et al. JAMA, Vol. 324, No. 6, 603-605, 2020).

The described conditions are summarized under the term “Post-COVID-19 syndrome” or “Long-COVID-19”, hereinafter referred to as Post-COVID.

Persons with post viral fatigue syndrome are complaining of physical and mental exhaustion, including reduced activity, reduced motivation, poor memory, concentration difficulties, unrefreshing sleep, emotional lability and in some cases depression. Recommendations for these persons are usually limited to behavioral advices, such as keeping exercise low at the beginning and increasing activity slowly, maintaining a routine of daily activities (eating, sleeping etc.) to support recovery.

Practical medical treatment strategies of patients suffering from Long-COVID are summarized in Deutsches Ärzteblatt 2020; 49, 117 and are based on primary care recommendations of Long-Covid as described by Greenhalgh, T.; Knight M., A Court C.; BMJ 2020; 370, 3026.

Moscatelli, F. et al., (Nutrients 2021; 13, 976-988) discuss the role of nutritional inventions and highlight the fact that strong data from clinical trials are needed to support any such assumption. Adequate nutrition is required to support the immune cell function by allowing to engage robust responses to pathogens. The micronutrients with the strongest evidence for immune support are vitamins C, D and zinc.

Creatine is a methylguanidinoacetic acid usually available from animal-based foods and/or produced naturally in the body from the amino acids arginine, glycine and methionine. Endogenous creatine synthesis provides about half of the daily need. The remaining amount of creatine needed to maintain normal tissue levels of creatine is obtained in the diet from animal based products such as fish or meat or from dietary supplements. Creatine plays a vital role in the energy metabolism in every cell of the body. It mainly serves as a metabolic intermediary of energy transfer by facilitating the recycling of ATP, the source of energy for use and storage at the cellular level. Thus, creatine is found in high concentrations in organs with high energy turnover, with ˜95% of the human body's creatine stores in the skeletal muscle and the remaining 5% in the brain, liver, kidney, and testes (McCall, W., Persky, A M. Pharmacokinetics of creatine. Subcell Biochem 2007, 46, 261-273; Bonilla, D. A. et al., Metabolic Basis of Creatine in Health and Disease: A Bioinformatics-Assisted Review. Nutrients 2021, 13, 1238; Brosnan, M. E. et al., The role of dietary creatine. Amino Acids 2016, 48, 1785-1791; Harris, R. Creatine in health, medicine and sport: An introduction to a meeting held at Downing College, University of Cambridge, July 2010, Amino Acids 2011, 40, 1267; Harris, R. C. et al. Elevation of creatine in resting and exercised muscle of normal subjects by creatine supplementation. Clin. Sci. 1992, 83, 367-374; Kreider, R. B.: Stout, J. R. Creatine in Health and Disease. Nutrients 2021, 13, 447; Ostojic, S. M.; Forbes, S. C. Perspective: Creatine, a Conditionally Essential Nutrient: Building the Case. Adv. Nutr. 2021, 00, 1-4).

The administration of creatine has therefore been considered as a supportive measure for the treatment of a variety of diseases. In particular Ostojic. S. M. et al. for example describes a dietary treatment of chronical fatigue syndrome (CFS) comprising guanidinoacetic acid (GAA) (Nutrients 2016, 8, 72). The potency of creatine in the treatment of post-viral fatigue syndrome (PVFS) is discussed in Ostojic, S. M. Nutrients 2021, 13, 503; Kreider R. B. et al., Nutrients 2021, 13, 447 and Ostojic, S. M. Nutritional Neuroscience, An International Journal of Diet, Nutrition and the Nervous System, vol. 25, no. 4, 2022, 884-885.

S. Marinari et al., Effects of neutraceutical diet integration, with coenzyme Q10 (Q-Ter multicomposite) and creatine, on dyspnea, exercise tolerance, and quality of life in COPD patients with chronic respiratory failure, multidisciplinary respiratory medicine 2013, 8:40 is a study on dietary supplementation with creatine and coenzyme Q10. COPD is a respiratory disease, while PVFS and in particular post-COVID is a neurological disease. S. M. Ostojic, Can creatine help in pulmonary rehabilitation after COVID-19?. Ther. Adv. Respir. Dis. 2020, Vol. 14:1-2 raises the question whether creatine supplementation could backup pulmonary rehabilitation in COVID-19. Z. Naureen et al., European Review for Medical and Pharmacological Sciences, 2021, 25 (1 Suppl): 67-73 gives a proposal of a food supplement for the management of post-COVID syndrome. Therein a food supplement composition comprising vitamin C, acetyl-L-carnitine, hydroxytyrosol/olive polyphenols, thiamine, vitamin B6, folic acid, vitamin D3 and vitamin B12 is suggested. L. Barrea et al., Nutrients 2022, 14, 1305 gives dietary recommendations for post-COVID-19 syndrome. Regarding post-COVID-19 fatigue syndrome the authors assume that there is evidence that the deficiency of some nutrients seems to be important including vitamin C, vitamin B group, sodium, magnesium, zinc, folic acid, L-carnitine, L-tryptophane, essential fatty acids and coenzyme Q10.

Starting from this the problem to be solved by the present invention is to improve the recovery from post viral fatigue syndrome (PVFS) caused e.g. by SARS-COV-2 virus, particularly of patients suffering under PVFS associated with mental fatigue, reduced motivation, reduced activity, or concentration difficulties.

### DESCRIPTION OF THE INVENTION

The problem is solved by administration of creatine in patients in need thereof. Creatine supports the recovery from PVFS, particularly when PVFS is caused by SARS-COV-2 virus. Especially the mental status of patients suffering under PVFS can be improved by administration of creatine, and/or the time to exhaustion can be increased. The mental status of patients comprises parameters such as mental fatigue, reduced motivation, and concentration difficulties.

Post-viral fatigue syndrome (PVFS) is a perplexing long-term neurological disorder. PVFS is in particular characterized by an inability to participate in routine activities that were possible before becoming ill, lasting for more than six months and accompanied by fatigue, post-exertional malaise and unrefreshing sleep. PVFS-related symptoms are in particular often found after infection with a member of the corona virus family (SARS-COV2) leading in many cases to Post-COVID fatigue syndrome.

The inventors of the present application have surprisingly found that creatine is effective for improving conditions associated with the mental status of a patient such as mental fatigue, reduced motivation and concentration difficulties as well as to increase time to exhaustion in patients suffering under PVFS. Thereby it was found that the effect is supported by a strong enrichment of creatine in the thalamus, grey matter and particularly white matter of the brain in patients suffering under Post-COVID (see FIG. 1). The enrichment is achieved by the supplemental administration of creatine in patients in need thereof.

This is surprising, since it was assumed previously that creatine cannot cross the blood-brain barrier. Within the framework of the present invention it has been found, however, that seemingly long-COVID or Post-COVID causes changes in the blood-brain barrier so that a creatine uptake and enrichment in brain areas such as the thalamus, grey matter and white matter, specifically in post-COVID situations was observed.

Further, the inventors of the present application found that Long-COVID patients, without supplementation of creatine, show only low levels of creatine in the brain. Within the framework of the present invention it was found that the concentrations of total creatine in the brain, and in particular in the thalamus, in the white matter and in the grey is significantly decreased in Long-COVID patients compared to the reference values of the general population. Thus, without being bound to any theory, it is assumed that one of the effects and/or causes of Long-COVID is depletion of creatine in brain areas and, thus, decrease of the creatine level in such brain areas. By the inventive finding that creatine is able to cross the blood-brain barrier in Long-COVID patients the creatine levels in the brain areas can be enriched and/or increased by the addition of creatine.

It is particular surprising that while the concentration of total creatine in the brain was found to be reduced in Long-COVID patients compared to reference values for the general population on the one hand, on the other hand, a significant enrichment of creatine in the brain, in particular in the thalamus, grey matter and/or white matter was found in Post-COVID patients, while no or only very little increase of creatine in the brain of about max. 5% is seen in normal healthy population after creatine supplementation. Seemingly, Long-COVID effects a depletion of brain creatine and at the same time alters the properties of the blood-brain barrier, so that supplemented creatine can pass, and results in a significant increase in creatine in the brain after creatine supplementation. Surprisingly, according to the invention an enrichment of creatine in the brain after creatine supplementation was found indicating that creatine in Long-COVID patients can pass the blood-brain barrier. It was in particular found that supplementation of creatine alone, i.e. supplementation of creatine without any transporters or auxiliary agents known for altering the blood-brain barrier results in enrichment of creatine in the brain.

Thus, according to the present invention, brain-related symptoms of Long-COVID such as mental fatigue, reduced motivation, concentration difficulties and/or time to exhaustion, all being neurological conditions, can be treated.

One of the difficulties associated with finding suitable treatments of PVFS and in particular of Post-COVID is the variety, multiplicity, diversity and vagueness of symptoms associated with Post-COVID and at the same time the uncertainty about the causes of the symptoms. This complicates both the treatment of Post-COVID and a forecast of which agents or treatments might function in the treatment of Post-COVID. Manifestations of Post-COVID occur as many different conditions and symptoms including pulmonary conditions, neurological symptoms and conditions such as headaches, nasal smell disorders, impaired sense of taste, dizziness, mental confusion, disorientation, and other impairments, neuropsychiatric disorders, strokes, gastrointestinal symptoms such as nausea, loss of appetite, vomiting, diarrhea; cardiovascular diseases including myocarditis, cardiac insufficiency, cardiac failure, and thromboembolic events; renal insufficiency; dermatological manifestations. In particular with regard to long-term effects, no uniform clinical picture can be defined, and the underlying mechanisms are not clear. Post-COVID patients report quite different symptoms which persist over weeks and even over months. Quite often reported complaints or symptoms include fatigue, weariness, mental fatigue, exhaustion, impaired resilience, memory problems, sleep problems, muscle weakness, muscle pain and psychic problems such as depressive symptoms and anxiety. Other symptoms reported include deterioration of pulmonary function, deterioration of lung function, deterioration of kidney function and heart muscle inflammation. This list is in no way conclusive, however, shows the diversity and variety of conditions and symptoms associated with Post-COVID. What is even more unknown up to date are the causes initiated by COVID or Post-COVID for those conditions and symptoms. The provision of suitable treatments of Post-COVID is therefore difficult, since mutual applicability of treatments known for similar symptoms does not exist.

According to the present invention it was now surprisingly found that a pharmaceutical composition comprising creatine or physiologically acceptable derivatives and/or salts and/or adducts thereof is effective for the treatment of specific conditions selected from the group of mental fatigue, reduced motivation, concentration difficulties associated with Post-COVID and in addition suitable to increase time to exhaustion in Post-COVID patients. As outlined, the causes of the numerous various symptoms associated with Post-COVID are not known and, thus, an effective treatment is not predictable. Further, it appears that not only the symptoms are quite numerous but also the conditions causing the various symptoms. In the tests and experiments underlying the present invention it was now found that provision of creatine specifically improves the mental status of Post-Covid patients and in particular improves the status of mental fatigue, reduced motivation and concentration difficulties. In particular with regard to mental fatigue, significant improvement was found.

Therefore, a first embodiment of the invention is a pharmaceutical composition comprising creatine or physiologically acceptable derivatives and/or salts and/or adducts thereof for use in the treatment of PVFS according to claim 1, in particular of PVFS caused by SARS-COV-2.

A second embodiment of the invention is the use of creatine or physiologically acceptable derivatives and/or salts and/or adducts thereof as dietary supplement or as a supplement for preparation of a diet supporting the recovery from PVFS, in particular when the PVFS is caused by SARS-COV-2 and when the PVFS is associated with symptoms selected from the group of mental fatigue, reduced motivation, or concentration difficulties. Thus, particularly the mental situation of subjects suffering under Post-COVID fatigue (PCFS) can be improved by the administration of creatine.

A further embodiment of the present invention is the administration of creatine in combination with glucose. Glucose supports the uptake of creatine in the brain compared to the ingestion of creatine alone. While it has been found according to the present invention that creatine administered alone can surprisingly cross the blood-brain barrier of Post-COVID patients and, thus, is enriched in brain areas, it was additionally found that the uptake of creatine in the brain can be further enhanced by supplemental administration of glucose. Surprisingly the time to exhaustion of Post-COVID patients can be increased by administration of glucose in addition to creatine compared to the sole administration of creatine, although the time to exhaustion decreases by the sole administration of glucose.

Glucose is preferably administered in combination with creatine. This means preferably within one hour before and one hour after the administration of creatine. Particularly preferred within 30 minutes, 15 minutes, 10 minutes or 5 minutes before and 30 minutes, 15 minutes, 10 minutes or 5 minutes after creatine ingestion. Most preferable creatine and glucose are administered together. The daily dose of glucose administered in combination with creatine is preferably in the range of 1 g to 10 g, particularly between 2 g and 7 g or 5 g. The daily glucose dose can be administered once a day, during breakfast for example, or spread over 2, 3, 4 or 5 times a day.

In a further embodiment of the invention the pharmaceutical composition or the dietary supplement comprising creatine or physiologically acceptable derivatives and/or salts and or/adducts thereof is used for the treatment of PVFS in combination with pulmonary rehabilitation and in particular with breathing exercises. As used herein the term breathing exercises includes physical exercises and also includes pulmonary rehabilitation. Pulmonary rehabilitation is preferably performed in accordance with Wang T. J. et al., Am J Phys Med Rehabil, 2020 June 11 (DOI 10.1097/PHM.0000000000001505/PMCID: 7315835). Pulmonary rehabilitation is tailored to each individual patient and includes for example modified segmental breathing, breathing exercises for strengthening pulmonary muscles, bed mobility exercise, stretching, gymnastics, and/or walking. The extent of the exercises is low at the beginning and is slowly increased without exposing the patient to higher efforts. The exercise frequency is e.g. 2 to 4 times per day for 10 to 15 minutes. The exercise time can be increased slowly. Pulmonary rehabilitation measures, such as training of the respiratory musculature and the muscles of exhalation by breathing exercises should be started as soon as possible, provided that the patient's state of health permits this. The rehabilitation is usually started within 20 weeks, preferably within 12 weeks, most preferably within 6 weeks, after infection has subsided.

The pharmaceutical composition of the first embodiment and the composition used as dietary supplement of the second embodiment are referred to herein after also as creatine composition, creatine comprising composition or composition comprising creatine. The term “creatine composition”, “creatine comprising composition” or “composition comprising creatine” comprise also physiologically acceptable creatine derivatives, creatine salts and/or creatine adducts if not expressly stated otherwise.

The creatine comprising compositions of the present invention are particularly useful to support recovery from fatigue syndrome after virus infection such as SARS-COV-2 infection, particularly when the post viral fatigue syndrome is associated with mental fatigue, reduced motivation, and concentration difficulties. However, PVFS is one of the most occurring condition of Post-COVID. A fatigue syndrome often persists for weeks, months or even longer in patients with an overcome SARS-COV-2 infection and can compromise general health significantly.

Several acute and chronic viral infections which cause PVFS, include coronaviruses (e.g. SARS, MERS, SARS-COV-2), Epstein-Barr virus, cytomegalovirus, and coxsackieviruses. A post viral fatigue syndrome can persist for weeks or months. After a SARS-COV-2 infection, for example, PVFS can persist up to a year or even longer.

The recovery of patients suffering under PVFS can be surprisingly improved by administration of creatine, in particular in combination with glucose. Creatine is also known as methylguanidinoacetic acid, which is naturally occurring in the body of animals and humans. Other names for creatine are N-(Aminoiminiomethyl)-N-methyl-glycine or N-Methyl-N-guanylglycine. Creatine is further available from animal-based foods or in higher amounts as food supplement. In food supplements preferably creatine monohydrate is used, which can be prepared in very high purity.

Beside creatine, also physiologically acceptable creatine derivatives can be used in accordance with the invention. Such creatine derivatives can be natural occurring compounds, such as creatine phosphate, or pro-drugs of creatine, which are able to release creatine under physiological conditions, such as creatine esters. In the context of the present invention, guanidinoacetic acid (GAA) is also included in the group of suitable creatine derivatives. Physiologically acceptable creatine derivatives are preferably selected from the group consisting of creatine, creatine hydrates, creatine esters or amides, including creatine C₁-C₅-alkyl esters, N—C₁-C₅-alkyl amides, creatine phosphate, creatinol-O-phosphate or a mixture thereof.

Suitable creatine salts, creatine adducts, salts of physiologically acceptable creatine derivatives and adducts of the physiologically acceptable creatine derivatives are preferably selected from the group consisting of the corresponding acetates, citrates, maleates, fumarates, tartrates, malates, pyruvates, ascorbates, succinates, aspartates, lactates, oxalates, formates, benzoates, phosphates, sulfates, chlorides, hydrochlorides, of the corresponding potassium, sodium, calcium, magnesium salts, of the corresponding L-carnitine, acetyl-L-carnitine, taurine, betaine, choline, methionine adducts or a mixture thereof.

Treatment with creatine can already begin during the virus infection, preferably within about three months (12 weeks) after infection. The duration of supplementation of creatine lasts normally between 1 week and 18 months or longer, preferably between 1 and 12 months, in particular between 3 and 8 months depending on the symptoms of the subject in need thereof.

The amount of creatine to be administered is in the range of 3 g to 30 g per day. Preferably the dosage is in the range of 7 g to 25 g, most preferred between 8 g and 20 g, which is higher than commonly recommended for sportsmen or sportswomen.

Preferably the administration of creatine is divided in an accumulation phase and a maintaining phase, wherein the daily dose of creatine in the composition is in the range of 10 g to 30 g in the initial accumulation phase and in the range of 7 g to 15 g for the subsequent maintaining phase. The accumulation phase has a duration of up to 3 weeks, preferably between 3 and 14 days, particularly between 5 and 10 days and the maintaining phase has a duration of 1 week to 18 months, preferably between 2 and 12 months, in particular between 3 and 8 months.

The accumulation phase is usually the initial phase. However, additional accumulation phases, for example with a duration between 1 day and 7 days, may be integrated into the maintaining phase.

The daily creatine dose can be administered once a day, during breakfast for example, or spread over 2, 3, 4 or 5 times a day.

If a combination of creatine and glucose is administered, the creatine to glucose weight ratio is preferably in the range of 1:5 to 5:1, more preferably between 1:3 and 3:1, particularly from 1:1 to 1:3.

If the administered creatine composition comprises a creatine derivative, pro drug, adduct or salt, the amount of the creatine moiety contained therein is decisive for the daily dosage within the ranges described above.

The creatine composition described herein may be preferably administered orally, in form of tablets, coated tablets, capsules, granules or powders.

In particular, granulates or powders comprising the creatine composition are used as aqueous suspension or in water-soluble form. The solubility of pure creatine and several creatine derivatives is low. Creatine for example has a solubility of 17 g/L (at 20° C.). Creatine and derivatives thereof with low solubility can be used in form of an aqueous suspension. A disadvantage of an aqueous suspension is often, that it segregates before ingestion. Therefore water-soluble granules or powders are usually preferred. To increase the water solubility of creatine or creatine derivatives water soluble salts or adducts thereof may be used. To increase the water-solubility particularly the use of acids or complexing agents may be useful to provide the corresponding creatine salts or creatine derivative salts. Examples for suitable acids, including carboxylic acids, and complexing agents are selected from the group malic acid, aspartic acid, ascorbic acid, succinic acid, pyruvic acid, fumaric acid, gluconic acid, alpha-ketoglutaric acid, oxalic acid, acetic acid, formic acid, sulfuric acid, hydrochloric acid, L-carnitine, acetyl-L-carnitine, taurine, betaine, choline, and lipoic acid. Peptides and amino acids may be also useful to increase the solubility of creatine and creatine derivatives. Further sodium, potassium, calcium and magnesium salts may be used to increase the water-solubility of creatine or creatine derivatives.

The molar ratio of creatine or the creatine derivatives and the acids or complexing agents mentioned above is usually in the range of 5:1 to 1:5, preferably in the range of 2:1 to 1:2, in particular in the range of 1.3:1 to 1:1.3.

The granules and powders can also be used for preparing foodstuff supporting the recovery from PVFS, in particular by improving the mental status of patients suffering under PVFS.

The creatine composition used in accordance with the present invention may be applied further in tablet form.

The creatine composition may be tableted as such or in form of a formulation comprising excipients. Suitable excipients are for example pharmacologically inactive ingredients, such as binders, fillers, antioxidants, preservatives, stabilizers, anti-caking agents, lubricants, disintegrants, flavors, pigments etc.

As binder or filler a wide variety of compounds may be used. Dibasic calcium phosphate; saccharides, including lactose and sucrose; polysaccharides and derivatives thereof, including starches, cellulose, modified cellulose, and cellulose ethers, such as hydroxypropyl cellulose or hydroxyethyl cellulose; microcrystalline cellulose; sugar alcohols, such as xylitol, sorbitol or mannitol; peptides, such as gelatin; polymers. e.g. polyvinylpyrrolidone, polyethylene glycol, etc. are common binders or fillers for tablets.

Typical suitable preservatives are for example cysteine, methionine, citric acid, sodium citrate, tetrazines or synthetic preservatives like parabens, including methyl paraben and propyl paraben, and benzoic acid. Suitable antioxidants may be selected from group of vitamin A, vitamin C, vitamin E, retinyl palmitate, and selenium.

Lubricants and anti-caking agents reduce the adhesion in granulates or powders and thus prevent sticking to tablet punches. They are also used to help protect tablets from sticking. The most commonly used anti-caking agents is magnesium stearate, stearic acid, or stearin, but also other magnesium or calcium salts of fatty acids may be used instead or in addition. Common mineral lubricants for example are talc or silica.

Disintegrants expand and dissolve when wet causing a tablet to break apart in the digestive tract, or in specific segments of the digestion process, releasing the ingredients for absorption by the body. Examples of disintegrants include crosslinked polymers like crosslinked polyvinylpyrrolidone (crospovidone) and crosslinked sodium carboxymethyl cellulose (croscarmellose sodium). Other suitable disintegrants are for example modified starch or sodium starch glycolate.

Flavors can be used to mask unpleasant tasting tablet ingredients. Further the ingredients may increase the patients' acceptance of the tablets. Flavorings may be natural, e.g. fruit extracts, or artificial. Natural extracts of vanilla, peach, apricot, raspberry, mint, anise or cherry can be used as flavors, for example. Antacid compounds or cough syrup would be also suitable.

Suitable pigments and colours are for example food colourants.

Tablet coatings protect tablet ingredients from deterioration by moisture in the air and make large or unpleasant-tasting tablets easier to swallow. For most coated tablets, a cellulose ether, particularly hydroxypropyl methylcellulose (HPMC) film coating is used. But other coating materials are also useful, for example synthetic polymers, shellac, plant fibers, waxes, fatty acids or polysaccharides. Capsules are coated usually with gelatine.

Particularly useful coatings for tablets related to the invention disclosed herein are crosslinked sodium carboxymethyl cellulose (croscarmellose sodium), silicified microcrystalline cellulose, agglomerated anhydrous lactose and lactitol monohydrate.

Preferred tablets comprise at least
a) 10 wt.-% to 100 wt.-%, preferably 30 wt.-% to 99 wt.-% creatine, creatine derivatives or salts or adducts thereof;
b) 0 to 80 wt.-% carbohydrates, including preferably glucose;
c) 0 to 20 wt.-% anti-caking agents selected from fatty acids or fatty acid salts, particularly magnesium stearate;
d) 0 to 20 wt.-% acidifier, preferably citric acid;
e) 0 to 20 wt.-% fatty acids, preferably coconut oil;
f) 0 to 5 wt.-% flavors.

Preferred capsules comprise at least
a) 10 wt.-% to 95 wt.-%, preferably 30 wt.-% to 80 wt.-% creatine, creatine derivatives or salts or adducts thereof
b) 0 to 80 wt.-% carbohydrates, including preferably glucose;
c) 0 to 20 wt.-% anti-caking agents selected from fatty acids or fatty acid salts, particularly magnesium stearate,
d) 0 to 5 wt.-% flavors;
e) 5 to 90 wt.-% gelatin.

The described ingredients may be useful not only in tablets but also in granules or powders comprising creatine or creatine derivatives or salts or adducts thereof. Particularly binders, fillers, antioxidants, preservatives, stabilizers, anti-caking agents, lubricants, disintegrants, flavors, pigments, and carbohydrates can be part of the creatine composition used in accordance with the present invention. Preferably the granules or powders comprises a combination of creatine and glucose.

Preferred granules or powders relating to the described invention comprise 10 wt.-% to 100 wt.-%, preferably 30 wt.-% to 99 wt.-% creatine, creatine derivatives or salts or adducts thereof.

Particularly preferred tablets, capsules, granules or powders comprising between 10 to 80 wt.-% glucose, preferably between 30 and 70 wt.-%. The preferred ratio of creatine:glucose in the compositions is in the range of 1:3 to 3:1, most preferred between 1:2 and 2:1.

Creatine and glucose can be formulated together e.g. in tablets, capsules, or granules, but the combination can also be administered in form of mixture of the compounds. e.g. in powders. A further possibility is a separate intake of creapure and glucose, preferably with a time lag of below 2 hours, most preferably below 1 hour or even below 30 minutes. Advantageously creatine and glucose are administered together. However, further preferred is the intake of creatine in a first step before glucose is administered in a second step.

In combination with creatine, creatine derivatives or salts or adducts thereof, anti-inflammatory drugs can be applied. Anti-inflammatory drugs are for example non-steroidal drugs, such as aspirin, ibuprofen, naproxen, diclofenac, celecoxib, mefenamic acid, etoricoxib, indomethacin etc. or steroids, such as corticosteroids, including cortisone, hydrocortisone and prednisone.

Further combinations of creatine, creatine derivatives or salts or adducts thereof, with neuroprotective drugs can be advantageous. Preferred neuroprotective agents include glutamate excitotoxicity inhibitors such as ginsenoside, riluzole, progesterone, estrogen, memantine, or simvastatin; stimulants such as caffeine; growth factors such as IGF-1, CNTF; nitric oxide synthase inhibitors; and caspase inhibitors or erythropoietin.

A combination of creatine, creatine derivatives or salts or adducts thereof, with drugs for the treatment of the virus infection is also possible, in particular if the administration of creatine is started during the acute phase of the virus infection. For the treatment of COVID-19 drugs of the group Lagevrio (molnupiravir), Olumiant 20 (baricitinib), tixagevimab/cilgavimab, Kineret (anakinra), Paxlovid (PF-07321332/ritonavir), Regkirona (regdanvimab), RoActemra (tocilizumab), Ronapreve (casirivimab/imdevimab), Veklury (remdesivir), Xevudy (sotrovimab) are available for example. For the treatment of influenza Rapivab (peramivir), Relenza (zanamivir), Tamiflu (oseltamivir phosphate), Xofluza (baloxavir marboxil) are suitable drugs. For 25 the treatment of adenoviruses cidofovir, ribavirin, ganciclovir, and vidarabine, are useful drugs.

Creatine, creatine derivatives or salts or adducts thereof are preferably used in combination with a pain-reducing, anti-inflammatory diet according to a preferred embodiment of the present invention. The preferred diet shall be provide all nutrients for normal energy metabolism and healthy function of the nervous system. A particularly suitable diet should comprise vitamins. minerals, unsaturated fatty acids, amino acids, secondary plant metabolites, including anti-oxidative agents, phytonutrients or essential or semi-essential nutrients.

For a pain-reducing, anti-inflammatory diet particularly vitamins of the group of vitamin, C, vitamin D, vitamin E, vitamin K and B vitamins (thiamine, riboflavin, nicotinamide, pantothenic acid, pyridoxine, biotin, folate, and/or vitamin B12) should applied in sufficient amounts.

Important minerals are selected from group of magnesium, calcium, potassium, sodium, copper, manganese, zinc, selenium, and boric acid/boron.

The vitamins and minerals should be present in the diet in sufficient quantities so that no deficiency symptoms occur. Appropriate recommendations for the daily amount of these nutrients are issued by the German Society for Nutrition (Deutsche Gesellschaft für Ernährung e.V., Referenzwerte für die Nährstoffzufuhr, 2. Auflage, 7. Aktualisierte Ausgabe, 2021).

A further group of useful nutrients for a pain-reducing, anti-inflammatory diet are unsaturated fatty acids, in particular omega-3 fatty acid; docosahexanoic acid (DHA), eicosapentenoic acid (EPA), alpha-lipoic acid and lecithine.

A preferred diet should be also rich on amino acids selected from the group of L-tyrosine, arginine and glycine. Additional amino acids for example theanine, cystine, taurine, or mixtures thereof are enriched in a preferred diet.

Further compounds that should be present in a pain-reducing, anti-inflammatory diet are N-acetyl-L-cysteine, gamma-amino-butyric acid (GABA). S-adenosylmethionine (SAMe), ubiquintol (coenzyme Q10), NADH, resveratrol, lutein, lycopene, choline, and carnitine.

Phytonutrients, including secondary plant metabolites, which are particularly useful in a pain-reducing, anti-inflammatory diet are for example antioxidants, anthocyanidines, flavonoids, flavones, isoflavones, catechins, anthocyanidins, isothiocyanates, carotenoids, allyl sulfides, polyphenols, resveratrol, lutein, or lycopene. Particularly preferred secondary plant metabolites are oligomeric procyanidins and oligomeric proanthocyanidins (OPCs).

For a preferred diet also plants, particularly herbs, spices, fruits, vegetables and legumes, or extracts, oils, powders, or compounds thereof can be used, for example from the group boswellia serrata, Curcuma longa, grape seeds (in particular with OPCs), devil's claw, or cat's claw. Further tomatoes, olive oil, green leafy vegetables, such as spinach, kale, and collards, nuts like almonds and walnuts, or fruits such as strawberries, blueberries, cherries, and oranges are regarded as anti-inflammatory foods. Fatty fish like salmon, mackerel, tuna, and sardines have also anti-inflammatory effects.

The recommended nutrients for a pain-reducing, anti-inflammatory diet can be supplied by selecting suitable foods or by addition of the respective components via nutrition supplements.

### DESCRIPTION OF THE FIGURES

FIG. 1: Changes in tissue creatine levels in patients suffering under Post-COVID. Left columns representing the group of patients receiving creatine, right columns representing the placebo group.

FIG. 2 depicts calculated Cohen's effect sizes of the administration of creatine in Post-COVID patients.

### WORKING EXAMPLES

### Example 1

A study was conducted to evaluate effectiveness and safety of replenishing creatine via dietary supplementation in patients suffering under Post-COVID after a SARS-CoV-2 infection. This study employed a parallel-group, randomized placebo-controlled double-blind design. The allocation ratio to experimental group (creatine) and control group (placebo) was set at 1:1. The eligibility criteria for patients to be included in the trial were: age 18-65 years, COVID-19 positive test within last 3 months (as documented by valid PCR or antigen test), moderate-to-severe fatigue and at least one of additional Post-COVID-19 related symptoms such as anosmia, ageusia, breathing difficulties, lung pain, body aches, headache, and concentration difficulties. Exclusion criteria were other pulmonary and cardiovascular conditions, and history of dietary supplement use during the 4 weeks before the trial commences.

The study was conducted in compliance with the Declaration of Helsinki (7th revision). The data presented until now were collected at FSPE Applied Bioenergetics Lab at the University of Novi Sad, from October 2021 to January 2022. The experimental (creatine) group received 4 grams of creatine monohydrate per day, while control group (placebo) received equivalent amount of inulin. The participants were asked to take the intervention one time per day during breakfast, with experimental or control powder stirred in 250 ml of lukewarm water and consumed immediately afterwards. Both interventions were similar in appearance, texture, and sensory characteristics. Creatine monohydrate was provided by Alzchem Trostberg GmbH (Trostberg, Germany). The duration of intervention was 6 months, and participants were asked to refrain from using any other dietary supplements during the trial. All outcome measures were determined at baseline (pre-administration) and after 3 and 6 months. The primary outcome was the change in creatine levels in brain at baseline and at 3-month and 6-month follow-up, respectively. The minimal sample size (n=12) was calculated using power analysis (G*Power 3.1.9.3, Heinrich-Heine-Universität Düsseldorf), with the effects size set at 0.50 (medium effect), alpha error probability 0.05, power 0.80 for two groups, and two measurements (3-months) and three measurements (6-months follow-up), respectively, of study outcomes.

The number of participants who were randomly assigned, received intended treatment, and were analyzed for the primary outcome at this time was twelve in total, six in experimental group and six in control group. The participants recruited reported no major side effects of either intervention so far.

Accumulation of creatine in skeleton muscles in Post-COVID patients.

Tissue levels of creatine were measured with proton magnetic resonance spectroscopy (1.5 T Avanto scanner, Siemens, Erlangen, Germany) using matrix head coil in circularly polarized mode, with metabolite spectra in the specific regions of skeletal muscle and brain (vastus medials muscle, thalamus, frontal, precentral, paracentral, and parietal white and grey matter) processed as previously described (Appl Physiol Nutr Metab. 2016 September, 41 (9): 1005-7).

### Statistical Methods:

Initially, data were analyzed with the Shapiro-Wilk test for the normality of distribution and Bartlett's test for the homogeneity of the variances. When homogenous variances were verified for normally distributed data, summary measures for interaction effect (time vs. intervention) were compared by two-way ANOVA with repeated measures. When non-homogenous variances were identified, data were compared using Friedmann's test. Post-hoc LSD test and Wilcoxon test were used to identify differences between individual sample pairs for 2-way ANOVA and Friedmann's test, respectively. The significance level was set at P≤0.05. Effect sizes after the intervention were assessed by Cohen statistics, with d≥0.8 indicating a large effect. The data were analyzed using the statistical package SPSS version 24.0 for Mac (IBM SPSS Statistics, Chicago, IL).

Changes in tissue creatine levels after 3 months in patients suffering under Post-COVID are summarized in Table 1. The Table shows changes in tissue creatine levels in white matter (brain), thalamus, vastus medialis and grey matter (brain) after intervention with 4 gram creatine per day for 3 months and 6 months compared to creatine level of the placebo group.

TABLE 1
Changes in tissue creatine level (mM)

| Total | Creatine | | | Placebo | | |
|---|---|---|---|---|---|---|
| creatine (mM) | Baseline | 3 months | 6 months | Baseline | 3 months | 6 months |
| Vastus | 28.2 ± 3.0 | 30.0 ± 4.2 | 30.6 ± 3.4 | 20.6 ± 1.9 | 21.1 ± 3.1 | 21.9 ± 4.4 |
| medialis muscle | | | | | | |
| Thalamus | 5.7 ± 0.5 | 6.3 ± 0.9 | 6.3 ± 1.0 | 5.9 ± 1.5 | 6.1 ± 0.8 | 6.0 ± 1.0 |
| Left frontal | 5.9 ± 0.6 | 6.2 ± 0.6 | 6.2 ± 0.5 | 5.8 ± 0.6 | 5.7 ± 0.6 | 5.7 ± 0.4 |
| white matter | | | | | | |
| Right frontal | 5.6 ± 0.8 | 6.6 ± 0.8 | 6.8 ± 1.0 | 5.7 ± 0.8 | 5.6 ± 0.7 | 5.6 ± 0.4 |
| white matter | | | | | | |
| Left frontal | 6.6 ± 0.4 | 6.9 ± 0.7 | 6.8 ± 0.7 | 6.2 ± 0.7 | 6.2 ± 1.1 | 6.2 ± 0.8 |
| grey matter | | | | | | |
| Right frontal | 6.5 ± 0.7 | 6.2 ± 0.3 | 6.2 ± 0.5 | 6.4 ± 0.7 | 6.3 ± 0.7 | 6.3 ± 0.6 |
| grey matter | | | | | | |
| Left precentral | 6.0 ± 0.6 | 6.2 ± 0.8 | 6.1 ± 0.7 | 5.8 ± 0.4 | 5.8 ± 0.3 | 5.9 ± 0.4 |
| white matter | | | | | | |
| Right precentral | 5.8 ± 0.5 | 5.8 ± 0.7 | 6.0 ± 0.7 | 5.8 ± 0.3 | 5.8 ± 0.3 | 5.9 ± 0.2 |
| white matter | | | | | | |
| Left precentral | 6.7 ± 0.6 | 7.3 ± 0.9 | 7.1 ± 0.7 | 6.5 ± 0.4 | 6.3 ± 0.6 | 6.4 ± 0.7 |
| grey matter | | | | | | |
| Right precentral | 6.8 ± 0.4 | 7.2 ± 0.5 | 7.0 ± 0.7 | 6.7 ± 0.4 | 6.8 ± 0.4 | 6.6 ± 0.4 |
| grey matter | | | | | | |
| Left parietal | 5.3 ± 0.6 | 6.0 ± 0.9 | 6.0 ± 0.6 | 5.2 ± 0.4 | 5.1 ± 0.4 | 5.0 ± 0.3 |
| white matter | | | | | | |
| Right parietal | 5.3 ± 0.7 | 6.9 ± 0.9 | 6.8 ± 1.0 | 5.1 ± 0.6 | 5.2 ± 0.8 | 5.3 ± 0.5 |
| white matter | | | | | | |
| Left mesial | 7.2 ± 0.9 | 8.0 ± 1.2 | 8.0 ± 1.0 | 7.0 ± 0.7 | 7.0 ± 0.8 | 7.0 ± 0.9 |
| grey matter | | | | | | |
| Right mesial | 7.1 ± 0.3 | 8.2 ± 1.3 | 8.1 ± 1.3 | 7.1 ± 0.4 | 7.2 ± 0.5 | 7.1 ± 0.3 |
| grey matter | | | | | | |

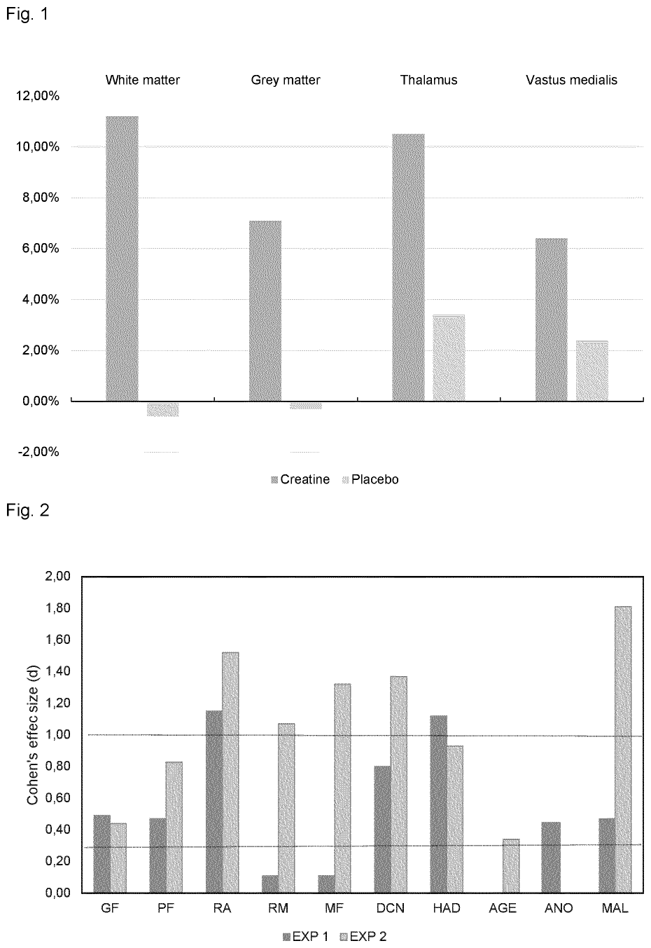

The differences in percent of the creatine level after three months and the corresponding baseline level in white matter (brain), grey matter (brain), thalamus, and vastus medialis muscle calculated from Table 1 are shown in FIG. 1. Left columns representing the group of patients receiving creatine, right columns representing the placebo group. For the values in white matter and grey matter average values are determined from Table 1.

In the randomized controlled trial, it was found that creatine is accumulated in brain and vastus medialis when administered for 3 months. The increased creatine level can be maintained for at least 3 further months by supplemental creatine administration. No changes in tissue creatine values were found in the placebo group.

The participants from the experimental group experienced an increase in tissue total creatine levels for all fourteen (14) locations evaluated in the study, with high increase at 6-month follow-up found in vastus medialis muscle (P=<0.01), left frontal white matter (P=0.01) and right parietal white matter (P=0.01). No changes in tissue creatine values were found in the placebo group throughout the trial. Two-way ANOVA with repeated measures revealed a significant difference (treatment vs. time interaction) between interventions in tissue creatine levels (P<0.05), with the creatine group superior to placebo to augment creatine levels at vastus medialis muscle, left frontal white matter, and right parietal white 6 matter. In addition, a strong interaction effect between interventions was reported for several other locations, including right frontal white matter, right paracentral grey matter, left parietal white matter, and left parietal mesial grey matter (P<0.20).

In addition, the Cohen's effect sizes for primary and secondary outcomes after creatine intake with strong effect sizes of creatine (d≥0.8) are demonstrated for increased brain levels in thalamus (0.82 at 3-month), right frontal white matter (1.25 at 3-month and 1.32 at 6-month), right paracentral grey matter (0.88 at 3-month), left parietal white matter (0.92 at 3-month and 1.17 at 6-month), parietal white matter (1.99 at 3-month and 1.74 at 6-month), left parietal messial grey matter (0.84 at 6-month), and right parietal messial grey matter (1.17 at 3-month and 1.06 at 6-month).

In sum, it was found that creatine is enriched in the brain of Post-COVID patients after supplementation with creatine and, thus, passes the blood-brain barrier.

Effect of creatine as nutrition supplement for Post-COVID patients.

The patient-reported outcomes for COVID-19-related signs and symptoms (e.g. headache, concentrating difficulties, anosmia, ageusia) were evaluated with VAS scale. Fatigue, comprising the conditions of the group general fatigue, physical fatigue, metal fatigue, reduced activity, and reduced motivation, was evaluated with a multidimensional fatigue inventory test (MFI-20 test, Smets E. M. et al., J. Psychosom. Res. 1995, 39 (3), 315). The results of the VAS scale survey and of the MFI-20 test are summarized in Table 2 and 3.

TABLE 2

| | Creatine | | | Placebo | | |
|---|---|---|---|---|---|---|
| | Baseline | 3 months | 6 months | Baseline | 3 months | 6 months |
| General fatigue | 12.0 | 11.3 | 12.7 | 12.0 | 11.8 | 12.8 |
| Physical fatigue | 13.5 | 12.7 | 13.2 | 12.5 | 12.7 | 12.8 |
| Mental fatigue | 12.3 | 11.2 | 10.5 | 11.0 | 11.3 | 12.2 |
| Reduced activity | 11.5 | 12.0 | 12.5 | 12.7 | 12.8 | 14.0 |
| Reduced motivation | 13.0 | 13.0 | 12.5 | 12.2 | 13.0 | 13.7 |

In the test performed, patients should report the score of the symptoms given in Table 2. Thus, the higher the score, the higher the respective symptom is experienced by the interviewed patient. As a consequence, the lower the value given, the better is the effect in mitigation of the respective symptom. As can be seen from Table 2, a quite significant improvement concerning mental fatigue is observed in the creatine group, which was not observed in the placebo group. Further, a certain improvement is seen for the symptom reduced motivation.

TABLE 3

| | Creatine | | | Placebo | | |
|---|---|---|---|---|---|---|
| | Baseline | 3 months | 6 months | Baseline | 3 months | 6 months |
| Headache | 3.8 | 0.8 | 0.5 | 4.4 | 2.2 | 0.0 |
| Concentrating | 4.0 | 1.5 | 0.0 | 2.6 | 0.9 | 0.2 |
| difficulties | | | | | | |
| Anosmia | 5.0 | 1.0 | 0.8 | 2.9 | 1.0 | 0.4 |
| Ageusia | 5.0 | 0.2 | 0.0 | 2.0 | 0.0 | 0.0 |

As can be seen in Table 3, considerable improvement of the condition concentrating difficulties can be achieved in the creatine group.

In addition the time to exhaustion of the patients was evaluated by an incremental test until exhaustion on a motorized treadmill. Speed and gradient of the treadmill are increased every third minute, starting at 2.7 km/h (1.7 miles per hour) at a 10% gradient and rising at stage 7 to 9.7 km/h (6 miles per hour) at 22% gradient (Will P. M. and Walter J. D., Am Heart. J., 1999 December, 138, 1033). The results of the treadmill evaluation are provided in Table 4.

TABLE 4

| | Creatine | | | Placebo | | |
|---|---|---|---|---|---|---|
| | Baseline | 3-months | 6-months | Baseline | 3-months | 6-months |
| Time to | 894 | 922 | 959 | 879 | 883 | 897 |
| exhaustion (sec) | | | | | | |

The time to exhaustion is significantly increased in patients suffering under post viral fatigue syndrome after a SARS-COV-2 virus infection. The time to exhaustion of the creatine group is increased 7.3% compared to 2.1% after placebo intake. More important, the creatine group achieves stage 6 in the treadmill test, wherein the placebo group remains at stage 5. The improved endurance is associated with a reduction in fatigue symptoms, particularly with reduced mental fatigue and reduced motivation compared to an increase in the placebo group. The reduced motivation worsens over the observation period in the placebo group compared to slight improvement in the creatine cohort. Smaller improvements are achieved by creatine administration compared to the placebo group relating to fatigue symptoms like physical fatigue, and reduced activity according to the results presented in Table 2. In addition, creatine induced no major side effects.

### Example 2

### Combination of Creatine Supplementation and Pulmonary Rehabilitation:

A second study was conducted to evaluate effectiveness and safety of replenishing creatine via dietary supplementation in patients suffering under Post-COVID after a SARS-COV-2 infection. Eight male and female Post-COVID patients (age 33.5±9.9 years, weight 72.3±14.5 kg, and height 168.6±11.0 cm; 4 women) with moderate fatigue and breathing difficulties or lung pain, lung malaise volunteered to participate in this randomized controlled trial. All patients were allocated in a double-blind parallel-group design to receive either 4 grams of creatine monohydrate per day plus breathing exercise (2-3 times per day for 10-15 min) (experimental group) or breathing exercise only (control group) during a 3-month intervention period.

Pulmonary rehabilitation is performed in accordance with Wang T. J. et al., Am J Phys Med Rehabil, 2020 June 11 (DOI 10.1097/PHM.0000000000001505/PMCID: 7315835). Pulmonary rehabilitation is tailored to each individual patient and includes for example modified segmental breathing, breathing exercises for strengthening respiratory muscles and muscles of exhalation, inspiratory muscle training, bed mobility exercise, stretching, gymnastics, and/or walking. The extent of the exercises is low at the beginning and is slowly increased without exposing the patient higher efforts.

Detailed information on experimental protocols and testing procedures used are as described in Example 1.

All volunteers completed the trial, with no participants reported any side effects of either intervention. The changes in study outcomes during the trial are depicted in Table 6. The increase in tissue total creatine levels in several brain locations and in vastus medialis muscle are provided in Table 5.

TABLE 5
Changes in tissue creatine level (mM)

| | Creatine + | | Breathing | |
|---|---|---|---|---|
| Total | Breathing Exercise | | Exercise | |
| creatine (mM) | Baseline | 3 months | Baseline | 3 months |
| Vastus medialis | 30.5 ± 3.5 | 34.4 ± 6.3 | 30.1 ± 12.7 | 28.5 ± 9.2 |
| muscle | | | | |
| Thalamus | 5.0 ± 1.1 | 5.9 ± 0.9 | 5.5 ± 0.1 | 5.5 ± 0.1 |
| Left frontal | 7.1 ± 1.2 | 8.2 ± 1.3 | 6.8 ± 0.2 | 6.7 ± 0.9 |
| white matter | | | | |
| Right frontal | 6.1 ± 1.5 | 6.8 ± 0.9 | 6.5 ± 2.4 | 5.6 ± 0.8 |
| white matter | | | | |
| Left frontal | 6.6 ± 0.7 | 7.3 ± 1.1 | 6.5 ± 0.3 | 5.7 ± 0.4 |
| grey matter | | | | |
| Right frontal | 6.6 ± 1.1 | 8.1 ± 0.5 | 7.9 ± 1.3 | 6.7 ± 1.0 |
| grey matter | | | | |
| Left precentral | 6.4 ± 0.5 | 6.6 ± 0.5 | 6.2 ± 0.1 | 6.4 ± 0.5 |
| white matter | | | | |
| Right | 6.1 ± 1.1 | 7.0 ± 1.3 | 5.0 ± 0.5 | 5.0 ± 0.5 |
| precentral | | | | |
| white matter | | | | |
| Left paracentral | 7.1 ± 0.9 | 8.5 ± 1.0 | 6.9 ± 0.1 | 5.7 ± 0.8 |
| grey matter | | | | |
| Right | 7.2 ± 1.2 | 8.5 ± 0.4 | 6.8 ± 1.6 | 6.0 ± 1.6 |
| paracentral | | | | |
| grey matter | | | | |
| Left parietal | 5.9 ± 2.1 | 6.7 ± 1.7 | 5.1 ± 0.8 | 5.1 ± 0.9 |
| white matter | | | | |
| Right parietal | 5.5 ± 1.1 | 5.7 ± 0.9 | 5.4 ± 0.7 | 4.6 ± 0.1 |
| white matter | | | | |
| Left mesial | 8.0 ± 1.8 | 9.8 ± 2.5 | 7.3 ± 0.2 | 6.5 ± 0.1 |
| grey matter | | | | |
| Right mesial | 8.2 ± 0.9 | 9.7 ± 1.5 | 6.0 ± 0.4 | 5.9 ± 0.2 |
| grey matter | | | | |

TABLE 6

| | Creatine + | | Breathing | |
|---|---|---|---|---|
| | Breathing Exercise | | Exercise | |
| | Baseline | 3 months | Baseline | 3 months |
| General fatigue | 11.5 | 12.3 | 11.8 | 12.8 |
| Physical fatigue | 12.3 | 12.2 | 13.3 | 14.5 |
| Mental fatigue | 11.8 | 11.5 | 11.8 | 11.5 |
| Reduced activity | 12.5 | 12.8 | 12.5 | 12.8 |
| Reduced motivation | 13.5 | 12.8 | 16.0 | 13.0 |

The participants from the creatine group experienced an increase in tissue total creatine levels for all fourteen (14) locations evaluated in our study, with significant increase at 3-month follow-up found in vastus medialis muscle (P=0.04), thalamus (P=0.03), right frontal grey matter (P=0.04), right precentral white matter (P=0.01), right paracentral grey matter (P=0.03), and left parietal mesial grey matter (P=0.01). The Cohen's effect sizes (d) for creatine increment at above six locations varied from 0.77 (vastus medialis muscle) to 1.76 (right frontal grey matter), implying a rather large effect of creatine monohydrate plus breathing exercise for muscle and brain creatine amplification. No elevation in total creatine levels was found for any location in the control group (except for a non-significant increase in left precentral white matter); furthermore, creatine levels significantly dropped in right frontal grey matter and left parietal mesial grey matter at 3-month follow-up in the control group (P<0.05). In addition, two-way ANOVA with repeated measures revealed significant differences for changes in total creatine levels between groups at four brain locations (P<0.05), with the participants from the experimental group were superior than the participants from the control group to augment brain creatine concentrations at left frontal grey matter, right frontal grey matter, right precentral white matter, and left parietal mesial grey matter.

Tissue total creatine concentrations at 3-month follow-up remained largely unresponsive to breathing exercise (or even further diminished from baseline levels), suggesting long-term disturbances in tissue bioenergetics after this complex condition.

### Example 3

Combination of creatine supplementation and glucose administration:

Fifteen male and female PCFS patients (age 39.7±16.0 years, weight 74.0±9.7 kg, height 173.9±8.8 cm; 9 women) with moderate fatigue and at least one of additional COVID-related symptoms (e.g. ageusia, anosmia, body aches, breathing difficulties, difficulties concentrating, headache, lung pain, malaise) volunteered to participate in this randomized controlled parallel-group interventional trial. All patients were allocated in a double-blind parallel-group design to receive a powder mixture of creatine monohydrate and glucose (experimental group 1; 8 grams of creatine (Creapure®) and 3 grams of glucose per day), creatine monohydrate (experimental group 2; 8 grams of creatine (Creapure®) per day) or glucose (control group; 3 grams of glucose per day) t.i.d. during an 8-week intervention interval. All participants refrained from using any other dietary supplements during the study. Detailed information about experimental protocols and testing procedures used in this study are described in Example 1.

All volunteers completed the trial, with no participants reported any side effects of either intervention. Changes in tissue creatine levels in patients suffering under Post-COVID are depicted in Table 7. The Table shows changes in tissue creatine levels in white matter (brain), thalamus, vastus medialis and grey matter (brain) after creatine+glucose intervention or creatine intervention compared to a control group (glucose administration).

TABLE 7

| Total | Creatine + Glucose | | Creatine | | Glucose | |
|---|---|---|---|---|---|---|
| creatine (mM) | Baseline | 8 weeks | Basline | 8 weeks | Baseline | 8 weeks |
| Vastus | 30.0 ± 6.7 | 32.3 ± 6.8 | 29.0 ± 5.6 | 30.4 ± 5.2 | 30.1 ± 12.7 | 28.5 ± 9.2 |
| medialis muscle | | | | | | |
| Thalamus | 5.0 ± 0.8 | 5.1 ± 0.8 | 5.1 ± 1.0 | 5.3 ± 0.9 | 5.5 ± 0.5 | 5.5 ± 0.7 |
| Left frontal | 6.9 ± 0.9 | 7.2 ± 0.7 | 6.7 ± 0.7 | 6.8 ± 0.9 | 5.8 ± 0.4 | 5.6 ± 1.0 |
| white matter | | | | | | |
| Right frontal | 6.6 ± 0.8 | 6.8 ± 0.9 | 6.7 ± 1.0 | 7.0 ± 1.1 | 5.6 ± 0.8 | 5.6 ± 0.8 |
| white matter | | | | | | |
| Left frontal | 6.6 ± 0.8 | 6.8 ± 0.9 | 6.6 ± 0.6 | 6.5 ± 0.5 | 6.8 ± 0.4 | 6.3 ± 0.9 |
| grey matter | | | | | | |
| Right frontal | 6.8 ± 1.0 | 7.4 ± 1.2 | 6.6 ± 1.1 | 6.7 ± 1.0 | 7.0 ± 0.9 | 7.0 ± 0.7 |
| grey matter | | | | | | |
| Left precentral | 5.9 ± 0.5 | 6.3 ± 0.7 | 6.4 ± 1.0 | 6.7 ± 0.8 | 5.6 ± 0.6 | 5.6 ± 0.5 |
| white matter | | | | | | |
| Right precentral | 6.2 ± 1.0 | 6.8 ± 1.0 | 6.8 ± 0.9 | 7.4 ± 1.1 | 5.4 ± 0.6 | 5.3 ± 0.7 |
| white matter | | | | | | |
| Left paracentral | 7.3 ± 1.1 | 7.9 ± 1.4 | 6.8 ± 0.8 | 6.9 ± 0.9 | 7.0 ± 0.9 | 6.9 ± 0.7 |
| grey matter | | | | | | |
| Right paracentral | 7.1 ± 1.3 | 7.7 ± 1.4 | 7.2 ± 0.8 | 7.6 ± 1.6 | 6.4 ± 0.7 | 6.2 ± 1.1 |
| grey matter | | | | | | |
| Left parietal | 5.2 ± 0.8 | 6.3 ± 1.4 | 7.5 ± 1.6 | 7.8 ± 1.3 | 5.4 ± 0.6 | 5.3 ± 0.5 |
| white matter | | | | | | |
| Right parietal | 5.1 ± 0.7 | 5.6 ± 0.6 | 7.6 ± 1.9 | 7.7 ± 1.9 | 5.2 ± 0.7 | 5.2 ± 0.7 |
| white matter | | | | | | |
| Left mesial | 8.7 ± 1.2 | 9.2 ± 1.2 | 8.1 ± 1.3 | 8.6 ± 1.1 | 7.6 ± 0.7 | 7.4 ± 0.6 |
| grey matter | | | | | | |
| Right mesial | 8.4 ± 1.1 | 8.9 ± 1.0 | 8.4 ± 1.4 | 9.2 ± 1.0 | 7.3 ± 0.6 | 7.0 ± 1.0 |
| grey matter | | | | | | |

Total creatine levels increased in all fourteen locations after an 8-week intervention in experimental group 1, with the levels significantly higher at eight locations as compared to baseline concentrations (P≤0.05), including the vastus medical muscle, thalamus, right frontal grey matter, left and right precentral white matter, right paracentral grey matter, left parietal white matter, and right parietal mesial grey matter. The Cohen's effect sizes (d) varied across locations, with medium or large effects of creatine monohydrate plus glucose demonstrated for creatine increase at right frontal grey matter (d=0.54), left precentral white matter (d=0.66), right precentral white matter (d=0.60), left parietal white matter (d=0.97), and right parietal white matter (d=0.77). Creatine concentrations significantly increased at three locations after an 8-week intervention in experimental group 2 (P≤0.05), including the vastus medical muscle, and left and right parietal mesial grey matter; the effects were above the threshold for medium effect in parietal mesial grey matter only (d=0.65). No significant changes in creatine levels were found in control group (glucose group) (P>0.05), and creatine levels dropped from the baseline levels in ten out of fourteen locations evaluated (71.4%). Two-way ANOVA with repeated measures (treatment vs. time interaction) revealed significant differences for changes in total creatine levels at right precentral white matter and left paracentral grey matter between the groups (P≤0.05), with experimental groups 1 and 2 outcompeted control group to elevate creatine levels at these two locations. A strong trend for superior creatine levels was also noted for other locations, such as the vastus medialis muscle, left precentral white matter, right paracentral grey matter, left parietal white matter, left parietal mesial grey matter, and right parietal mesial grey matter (P<0.20).

The patient-reported outcomes for COVID-19-related signs and symptoms (e.g. headache, concentrating difficulties, anosmia, ageusia) were evaluated with VAS scale. Fatigue, comprising the conditions of the group general fatigue, physical fatigue, metal fatigue, reduced activity, and reduced motivation, was evaluated with a multidimensional fatigue inventory test (MFI-20 test, Smets E. M. et al., J. Psychosom. Res. 1995, 39 (3), 315). The results of the VAS scale survey and of the MFI-20 test are summarized in Table 8 and 9.

TABLE 8

| | Creatine + Glucose | | Creatine | | Gluose | |
|---|---|---|---|---|---|---|
| | Baseline | 8 weeks | Baseline | 8 weeks | Baseline | 8 weeks |
| General fatigue | 12.2 | 11.6 | 12.6 | 12.0 | 12.0 | 11.6 |
| Physical fatigue | 13.8 | 13.0 | 12.2 | 11.4 | 12.8 | 12.6 |
| Mental fatigue | 13.0 | 12.8 | 13.4 | 12.2 | 11.4 | 11.0 |
| Reduced activity | 12.6 | 10.6 | 13.6 | 12.0 | 12.6 | 12.6 |
| Reduced motivation | 11.4 | 11.2 | 11.6 | 10.3 | 10.0 | 10.8 |

TABLE 9

| | Creatine + Glucose | | Creatine | | Glucose | |
|---|---|---|---|---|---|---|
| | Baseline | 8 weeks | Baseline | 8 weeks | Baseline | 8 weeks |
| Headache | 5.0 | 2.6 | 3.2 | 0.8 | 3.8 | 2.8 |
| Concentrating | 3.2 | 1.6 | 4.2 | 2.2 | 4.4 | 3.0 |
| difficulties | | | | | | |
| Anosmia | 0.2 | 0.4 | 0.0 | 0.0 | 2.6 | 1.0 |
| Ageusia | 0.2 | 0.0 | 0.0 | 0.2 | 2.2 | 0.6 |
| Malaise | 4.8 | 3.8 | 4.2 | 1.0 | 4.6 | 3.0 |

In addition the time to exhaustion of the patients was evaluated by an incremental test until exhaustion on a motorized treadmill as described in Example 1.

TABLE 10

| | Creatine + Glucose | | Creatine | | Gluose | |
|---|---|---|---|---|---|---|
| | Baseline | 8 weeks | Baseline | 8 weeks | Baseline | 8 weeks |
| Time to | 872 | 899 | 824 | 844 | 915 | 908 |
| exhaustion (sec) | | | | | | |

The time to exhaustion is significantly increased in patients suffering under post viral fatigue syndrome after a SARS-COV-2 virus infection. The time to exhaustion of the creatine group is increased 20 sec. in experimental group 2 (creatine group) at the post-administration (P=0.03). More important, by a combination of creatine and glucose intake the time to exhaustion increases to 27 sec. in the treadmill test, which is in contrary to the expectation considering that the tome to exhaustion worsens after glucose intake (−7 sec.). The improved endurance was accompanied by a significant reduction in fatigue scores for the reduced activity subdomain in experimental group 2 (P=0.008), with d=1.52 implying a large effect of creatine monohydrate for this condition. The improved endurance is further associated with improvements in mental fatigue and reduced motivation.

Several Post-COVID fatigue (PCFS)-related symptoms, including concentration difficulties and general malaise, were significantly reduced in experimental group 1 (creatine+glucose) at 8-week follow-up (P≤0.05); the Cohen's effect sizes (d) for reducing concentration difficulties was 0.80, suggesting a large effect of creatine monohydrate plus glucose for this outcome. In addition difficulties concentrating, and general malaise were significantly reduced in experimental group 2 at post-administration (P<0.05); the Cohen's effect sizes for these variables were above a threshold value of 0.80, suggesting a large effect of creatine monohydrate. The participants receiving control intervention experienced no significant changes in most variables at the 8-week follow-up (P>0.05).

### CONCLUSIONS

Creatine monohydrate administered for eight weeks with or without glucose can be recommended as a well-tolerated intervention to improve tissue bioenergetics and several clinical features in patients suffering from post-COVID fatigue syndrome. The effects of creatine monohydrate (with or without glucose) were superior compared to control intervention for increasing creatine levels at the skeletal muscle and several brain locations (including both white and grey matter), patient-reported symptoms, including the reduction of problems with concentration, and general malaise. Creatine monohydrate also improved time to exhaustion and diminished specific fatigue subdomains (e.g. reduced activity). Glucose additionally augmented the ability of creatine monohydrate to improve creatine levels in specific locations across the brain, but not for most patient-reported outcomes in post-COVID fatigue syndrome. However, the endurance is improved by combined intake of creatine and glucose despite the indifferent patient-reported outcomes.

The Cohen's effect sizes (d) in experimental group 1 (EXP1, creatine+glucose) and experimental group 2 (EXP2, creatine) for all outcomes evaluated and reproduced in Table 11 and FIG. 2. The effects are classified as small (d=0.2), medium (d=0.5, dashed line), and large (d≥0.8). Missing values are due to a statistical limitation in calculating effect sizes when pre and/or post values equal zero.

### Abbreviations

GF, general fatigue; PF, physical fatigue; RM, reduced motivation; RA, reduced activity; MF, mental fatigue; AGE, ageusia; ANO, anosmia; DCN, difficulties concentrating; and HAD, headache.

TABLE 11

| | Cohen'effect (d), | Cohen's effect (d), |
|---|---|---|
| | EXP 1 | EXP 2 |
| General Fatigue (GE) | 0.49 | 0.44 |
| Physical Fatigue (PF) | 0.47 | 0.83 |
| Reduced Motivation (RM) | 0.11 | 1.07 |
| Reduced activity (RA) | 1.15 | 1.52 |
| Mental Fatigue (MF) | 0.11 | 1.32 |
| Breathing Difficulties (BRD) | 0.34 | 0.96 |
| Ageusia (AGE) | | 0.34 |
| Anosmia (ANO) | 0.44 | |
| Difficulties concentrating (DCN) | 0.80 | 1.37 |
| Headache (HAD) | 1.12 | 0.93 |
| Malaise (MAL) | 0.47 | 1.81 |

1. Pharmaceutical composition comprising creatine or a creatine hydrate and/or a salt thereof for use in the treatment of post viral fatigue syndrome (PVFS) after a SARS-CoV-2 infection associated with conditions selected from the group of mental fatigue, reduced motivation and/or concentration difficulties.

2. The pharmaceutical composition of claim 1, wherein the PVFS is caused by a coronavirus infection or are symptoms of Post-COVID after a SARS-COV-2 infection.

3. The pharmaceutical composition of claim 1, for use to increase time to exhaustion in patients with mental fatigue and/or reduced motivation.

4. The pharmaceutical composition of claim 1, wherein the composition is used in combination with glucose, preferably to increase time to exhaustion of subjects suffering under Post-COVID fatigue syndrome (PCFS) after a SARS-COV-2 infection.

5. The pharmaceutical composition of claim 1, wherein the creatine salt is selected from the group consisting of the corresponding acetates, citrates, maleates, fumarates, tartrates, malates, pyruvates, ascorbates, succinates, aspartates, lactates, oxalates, formates, benzoates, phosphates, sulfates, chlorides, hydrochlorides, of the corresponding potassium, sodium, calcium, magnesium salts.

6. The pharmaceutical composition of claim 1, wherein the composition comprises creatine monohydrate.

7. The pharmaceutical composition of claim 1, wherein the daily dose of creatine in the composition is in the range of 7 g to 30 g.

8. The pharmaceutical composition of claim 1, wherein the daily dose of creatine in the composition is an accumulation dose in the range of 10 g to 30 g for an initial accumulation phase and a maintaining dose in the range of 7 g to 15 g for a subsequent maintaining phase, wherein the accumulation phase has a duration of up to 3 weeks and the maintaining phase has a duration of 1 month to 12 months.

9. The pharmaceutical composition of claim 1 used in combination with glucose.

10. The pharmaceutical composition of claim 9, whereby the glucose is for administration within 1 hour before and 1 hour after the administration of creatine.

11. The pharmaceutical composition according to claim 9, wherein the daily dose of glucose in the composition is in the range of 1 g to 10 g.

12. A method of administering a composition comprising creatine or a creatine hydrate and/or a salt thereof as dietary supplement or as a supplement for preparation of a diet supporting the recovery from PVFS after a SARS-COV-2 infection associated with conditions selected from the group of mental fatigue, reduced motivation, reduced activity and/or concentration difficulties.

13. The method of claim 12, wherein the PVFS is caused by a coronavirus infection or are symptoms of Post-COVID after a SARS-COV-2 infection.

14. The method of claim 12, wherein the composition increases the time to exhaustion of subjects suffering under Post-COVID fatigue syndrome (PCFS) after a SARS-COV-2 infection.

15. The method of claim 12, wherein the daily dose of creatine in the composition is in the range of 7 g to 30 g.

16. The method of claim 12, wherein the daily dose of creatine in the composition is an accumulation dose in the range of 10 g to 30 g for an initial accumulation phase and a maintaining dose in the range of 7 g to 15 g for a subsequent maintaining phase, wherein the accumulation phase has a duration of up to 3 weeks and the maintaining phase has a duration of 1 month to 12 months.

17. The method of claim 12, wherein the composition comprises creatine monohydrate.

18. The method of claim 12, wherein the composition is used in combination with glucose.

19. The method of claim 18, wherein glucose is administered within 1 hour before and 1 hour after the administration of creatine.

20. The method of claim 18, wherein the daily dose of glucose administered in combination with creatine is in the range of from 1 g to 10 g.

21. A method of treating a subject for recovery of post viral fatigue syndrome (PVFS) after a SARS-COV-2 infection associated with conditions selected from the group of mental fatigue, reduced motivation and/or concentration difficulties comprising administering creatine or a creatine hydrate and/or a salt thereof.

22. The method of claim 21, wherein the PVFS is caused by a coronavirus infection or are symptoms of Post-COVID after a SARS-COV-2 infection.

23. The method of claim 21, wherein administering the composition increases time to exhaustion in patients with mental fatigue and/or reduced motivation.

24. The method of claim 21, further comprising administering glucose in combination with the composition to increase time to exhaustion of subjects suffering under Post-COVID fatigue syndrome (PCFS) after a SARS-COV-2 infection.

25. The method of claim 21, wherein the creatine salt is selected from the group consisting of the corresponding acetates, citrates, maleates, fumarates, tartrates, malates, pyruvates, ascorbates, succinates, aspartates, lactates, oxalates, formates, benzoates, phosphates, sulfates, chlorides, hydrochlorides, of the corresponding potassium, sodium, calcium, magnesium salts.

26. The method of claim 21 comprising administering creatine monohydrate.

27. The method of claim 21, wherein the daily dose of creatine is in the range of 7 g to 30 g.

28. The method of claim 21, wherein the daily dose of creatine in the composition is an accumulation dose in the range of 10 g to 30 g for an initial accumulation phase and a maintaining dose in the range of 7 g to 15 g for a subsequent maintaining phase, wherein the accumulation phase has a duration of up to 3 weeks and the maintaining phase has a duration of 1 month to 12 months.

29. The method of claim 21, further comprising administering glucose in combination with the composition.

30. The method of claim 29, whereby the glucose is administered within 1 hour before and 1 hour after the administration of creatine.

31. The method of claim 29, wherein the daily dose of glucose is in the range of 1 g to 10 g.
